(19) United States
(12) Patent Application Publication
(10) Pub. No.: US 2011/0145945 A1
(43) Pub. Date: Jun. 16, 2011

(54) Nematode-Resistant Transgenic Plants

(75) Inventors: Dasharath Prasad Lohar, Cary, NC (US); Steven Hill, Cary, NC (US)

(73) Assignee: BASF Plant Science GmbH, Ludwigshafen (DE)

(21) Appl. No.: 13/060,061

(22) PCT Filed: Aug. 24, 2009
(86) PCT No.: PCT/EP2009/060886

§ 371 Date: Feb. 22, 2011

Related U.S. Application Data
(60) Provisional application No. 61/092,100, filed on Aug. 27, 2008.

(51) Int. Cl. A01H 1/00; A01H 5/00; A01H 5/10; C12N 15/82

(52) U.S. Cl. 800/279; 800/295; 800/298; 435/320.1; 800/312; 800/317.2; 800/314; 800/306; 800/320.3

(57) ABSTRACT

The invention provides nematode-resistant transgenic plants and seed comprising polynucleotides encoding Medicago truncatula cysteine cluster proteins which comprise no more than four cysteine residues in the respective mature peptides. The invention also provides methods of producing transgenic plants with increased resistance to soybean cyst nematode and expression vectors for use in such methods.

### FIELD OF THE INVENTION

The invention relates to enhancement of agricultural productivity through use of nematode-resistant transgenic plants and seeds, and methods of making such plants and seeds.

### BACKGROUND OF THE INVENTION

Nematodes are microscopic roundworms that feed on the roots, leaves and stems of more than 2,000 row crops, vegetables, fruits, and ornamental plants, causing an estimated $100 billion crop loss worldwide. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore parasitic on a large number of crop species. The cyst- and lesion-forming nematode species have a more limited host range, but still cause considerable losses in susceptible crops.

Parasitic nematodes are present throughout the United States, with the greatest concentrations occurring in the warm, humid regions of the South and West and in sandy soils. Soybean cyst nematode (Heterodera glycines), the most serious pest of soybean plants, was first discovered in the United States in North Carolina in 1954. Some areas are so heavily infested by soybean cyst nematode (SCN) that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. Nematode infestation, however, can cause significant yield losses without any obvious above-ground disease symptoms. The primary causes of yield reduction are due to underground root damage. Roots infected by SCN are dwarfed or stunted. Nematode infestation also can decrease the number of nitrogen-fixing nodules on the roots, and may make the roots more susceptible to attacks by other soil-borne plant nematodes.

The nematode life cycle has three major stages: egg, juvenile, and adult. The life cycle varies between species of nematodes. The life cycle of SCN is similar to the life cycles of other plant parasitic nematodes. The SCN life cycle can usually be completed in 24 to 30 days under optimum conditions, whereas other species can take as long as a year, or longer, to complete the life cycle. When temperature and moisture levels become favorable in the spring, worm-shaped juveniles hatch from eggs in the soil. Only nematodes in the juvenile developmental stage are capable of infecting soybean roots.

After penetrating soybean roots, SCN juveniles move through the root until they contact vascular tissue, at which time they stop migrating and begin to feed. With a stylet, the nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As female nematodes feed, they swell and eventually become so large that their bodies break through the root tissue and are exposed on the surface of the root.

After a period of feeding, male SCN, which are not swollen as adult females, migrate out of the root into the soil and fertilize the enlarged adult females. The males then die, while the females remain attached to the root system and continue to feed. The eggs in the swollen females begin developing, initially in a mass or egg sac outside the body, and then later within the nematode body cavity. Eventually the entire adult female body cavity is filled with eggs, and the nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the protective cysts for several years.

A nematode can move through the soil only a few inches per year on its own power. However, nematode infestation can spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. For example, U.S. Pat. Nos. 5,589,622 and 5,824,876 are directed to the identification of plant genes expressed specifically in or adjacent to the feeding site of the plant after attachment by the nematode. A number of approaches involve transformation of plants with double-stranded RNA capable of inhibiting essential nematode genes. Other agricultural biotechnology approaches propose to over-express genes that encode proteins that are toxic to nematodes.

Leguminous plants such as soybean and alfalfa develop specialized root nodules when infected by symbiotic soil bacteria of genus Rhizobium. Once established within nodules Rhizobia fix atmospheric nitrogen, making it available for use by the plant. Nitrogen fixation in nodules is important for agriculture due to essential role of nitrogen as a plant nutrient. Many plant genes, referred to as “nodulins”, are preferentially expressed in nodules. Nodulin genes encode a wide variety of proteins, including leghemoglobin, uricase, glutamine synthetase, sucrose synthase, and numerous other proteins of unknown function.

One class of Medicago trunculata (alfalfa) nodulin genes encodes small proteins that are enriched in the amino acid cysteine, termed “Cys-cluster proteins” of “CCPs”. One subclass of CCPs is characterized by an N-terminal signal sequence; a small, highly charged polar mature peptide; and a characteristic arrangement of four cysteine residues that form two disulfide bridges within the mature peptide. This subclass of CCPs is distinguished from other M. trunculata CCP subclasses by the number of cysteine residues: other CCPs contain six, eight, or ten cysteine residues in the mature peptide and are likely to form more than two disulfide bonds in the mature peptide. Other than the characteristic arrangements of cysteines that the members of each subclass share, the CCPs demonstrate relatively low levels of amino acid identity.

The disulfide bridge patterns of the M. trunculata CCP mature peptides that contain more than four cysteine residues are similar to those of the plant defensins, which are low molecular weight cysteine-rich antimicrobial and antifungal proteins. Plant defensins comprise eight cysteines that form four structure-stabilizing disulfide bridges. The three-dimensional structure of the plant defensins features a “cysteine-stabilized αβ” or “CSαβ” motif that is shared by toxins from insects, scorpions, honeybees and spider venoms. The short-chain toxins such as scorpion toxin bind to either K⁺or C1⁻channels.

U.S. Pat. Nos. 6,121,436; 6,316,407; and 6,916,970 disclose the M. trunculata defensins AFP1 and AFP2. The AFP1 gene was transformed into potato under control of the constitutive FMV promoter, and the resulting transgenic plants demonstrated increased resistance to the fungus Verticillium dahliae both in the greenhouse and in field testing. (Gao, et al. (2000) Nat. Biotechnol. 18, 1307). Notwithstanding these positive results, no transgenic potato comprising a transgene encoding the AFP1 defensin has been commercialized to date.

U.S. Pat. Nos. 6,911,577 and 7,396,980 disclose plant genes encoding defensins from Oryza sativa, Zea mays, Triticum aestivum, Glycine max, Beta vulgaris, Hedera helix, Tulipa fosteriana, Tulipa gesneriana, Momordica charantia, Nicotiana benthamiana, Taraxacum kok-saghyz, Picramnia pentandra, Amaranthus retroflexux, Allium porrum, Cyamopsis tetragonoloba, Brassica napus, Vernonia mespilifolia, Parthenium argentatum, Licania michauxii, Ricinus communis, Eucalyptus grandis, Vitis vinifera, and Arachis hypogaea. The plant defensin genes disclosed in U.S. Pat. Nos. 6,911,577 and 7,396,980 are purported to confer resistance to parasites, including nematodes.

To date, no genetically modified plant comprising a transgene capable of conferring nematode resistance has been deregulated in any country. Accordingly, a need continues to exist to identify safe and effective compositions and methods for controlling plant parasitic nematodes using agricultural biotechnology.

### SUMMARY OF THE INVENTION

The present inventors have discovered that a transgene comprising a polynucleotide encoding a M. trunculata CCP mature peptide that contains no more than four cysteine residues can render soybean plants resistant to SCN infection. Accordingly, the present invention provides transgenic plants and seeds, and methods to overcome, or at least alleviate, nematode infestation of valuable agricultural crops.

In one embodiment, the invention provides a transgenic plant transformed with an expression vector comprising an isolated polynucleotide encoding at least one M. trunculata gene that encodes a CCP mature peptide that contains no more than four cysteine residues.

Another embodiment of the invention provides a seed produced by the transgenic plant described above. The seed is true breeding for a transgene comprising at least one M. trunculata gene that encodes a CCP mature peptide containing no more than four cysteine residues, and expression of the CCP gene or genes confers increased nematode resistance to the plant grown from the transgenic seed.

Another embodiment of the invention relates to an expression vector comprising a promoter operably linked to a polynucleotide encoding at least one M. trunculata CCP mature peptide that contains no more than four cysteine residues. Preferably, the promoter is a constitutive promoter. More preferably, the promoter is capable of specifically directing expression in plant roots. Most preferably, the promoter is capable of specifically directing expression in a syncytia site of a plant infected with nematodes.

In another embodiment, the invention provides a method of producing a nematode-resistant transgenic plant, wherein the method comprises the steps of: a) transforming a wild type plant cell with an expression vector comprising a promoter operably linked to a polynucleotide encoding at least one M. trunculata CCP mature peptide that contains no more than four cysteine residues; b) regenerating transgenic plants from the transformed plant cell; and c) selecting transgenic plants for increased nematode resistance as compared to a control plant of the same species.

### BRIEF DECRIPTION OF THE DRAWINGS

FIG. 1 shows the table of SEQ ID NOs assigned to corresponding genes and promoter.

FIG. 2 shows an amino acid alignment of MtCCP1 (SEQ ID NO:2), MtCCP3 (SEQ ID NO:4), MtCCP4 (SEQ ID NO:6), MtCCP5 (SEQ ID NO:8), MtCCP8 (SEQ ID NO:10), MtCCP2 (SEQ ID NO:12), MtCCP7 (SEQ ID NO:14), MtCCP9 (SEQ ID NO:16) and MtCCP6 (SEQ ID NO:18):. The alignment is performed in Vector NTI software suite (gap opening penalty=10, gap extension penalty=0.05, gap separation penalty=8).

FIG. 3 shows the global nucleotide percent identity between MtCCP genes: MtCCP1 (SEQ ID NO:1), MtCCP3 (SEQ ID NO:3), MtCCP4 (SEQ ID NO:5), MtCCP5 (SEQ ID NO:7), MtCCP8 (SEQ ID NO:9), MtCCP2 (SEQ ID NO:11), MtCCP7 (SEQ ID NO:13), MtCCP9 (SEQ ID NO:15) and MtCCP6 (SE ID NO:17). Pairwise alignments and percent identities were calculated using Needle of EMBOSS-4.0.0 (Needleman, S. B. and Wunsch, C. D. (1970) J. Mol. Biol. 48, 443-453).

FIG. 4 shows the global amino acid percent identity between MtCCP genes: MtCCP1 (SEQ ID NO:2), MtCCP3 (SEQ ID NO:4), MtCCP4 (SEQ ID NO:6), MtCCP5 (SEQ ID NO:8), MtCCP8 (SEQ ID NO:10), MtCCP2 (SEQ ID NO:12), MtCCP7 (SEQ ID NO:14), MtCCP9 (SEQ ID NO:16) and MtCCP6 (SEQ ID NO:18). Pairwise alignments and percent identities were calculated using Needle of EMBOSS-4.0.0 (Needleman, S. B. and Wunsch, C. D. (1970) J. Mol. Biol. 48, 443-453).

### DETAILED DESCRIPTION OF THE PREFERRED EMBODIMENTS

The present invention may be understood more readily by reference to the following detailed description and the examples included herein. Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used. As used herein, the word “or” means any one member of a particular list and also includes any combination of members of that list.

As defined herein, a “transgenic plant” is a plant that has been altered using recombinant DNA technology to contain an isolated nucleic acid which would otherwise not be present in the plant. As used herein, the term “plant” includes a whole plant, plant cells, and plant parts. Plant parts include, but are not limited to, stems, roots, ovules, stamens, leaves, embryos, meristematic regions, callus tissue, gametophytes, sporophytes, pollen, microspores, and the like. The transgenic plant of the invention may be male sterile or male fertile, and may further include transgenes other than those that comprise the isolated polynucleotides described herein.

As defined herein, the term “nucleic acid” and “polynucleotide” are interchangeable and refer to RNA or DNA that is linear or branched, single or double stranded, or a hybrid thereof. The term also encompasses RNA/DNA hybrids. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, it may be preferable to remove the sequences which naturally flank the coding region in its naturally occurring replicon.

The term “gene” is used broadly to refer to any segment of nucleic acid associated with a biological function. Thus, genes include introns and exons as in genomic sequence, or just the coding sequences as in cDNAs and/or the regulatory sequences required for their expression. For example, gene refers to a nucleic acid fragment that expresses mRNA or functional RNA, or encodes a specific protein, and which includes regulatory sequences.

The terms “polypeptide” and “protein” are used interchangeably herein to refer to a polymer of consecutive amino acid residues.

The terms “operably linked” and “in operative association with” are interchangeable and as used herein refer to the association of isolated polynucleotides on a single nucleic acid fragment so that the function of one isolated polynucleotide is affected by the other isolated polynucleotide. For example, a regulatory DNA is said to be “operably linked to” a DNA that expresses an RNA or encodes a polypeptide if the two DNAs are situated such that the regulatory DNA affects the expression of the coding DNA.

The term “promoter” as used herein refers to a DNA sequence which, when ligated to a nucleotide sequence of interest, is capable of controlling the transcription of the nucleotide sequence of interest into mRNA. A promoter is typically, though not necessarily, located 5′ (e.g., upstream) of a nucleotide of interest (e.g., proximal to the transcriptional start site of a structural gene) whose transcription into mRNA it controls, and provides a site for specific binding by RNA polymerase and other transcription factors for initiation of transcription.

The term “transcription regulatory element” as used herein refers to a polynucleotide that is capable of regulating the transcription of an operably linked polynucleotide. It includes, but not limited to, promoters, enhancers, introns, 5′ UTRs, and 3′ UTRs.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. In the present specification, “plasmid” and “vector” can be used interchangeably as the plasmid is the most commonly used form of vector. A vector can be a binary vector or a T-DNA that comprises the left border and the right border and may include a gene of interest in between. The term “expression vector” is interchangeable with the term “transgene” as used herein and means a vector capable of directing expression of a particular nucleotide in an appropriate host cell. The expression of the nucleotide can be over-expression. An expression vector comprises a regulatory nucleic acid element operably linked to a nucleic acid of interest, which is—optionally—operably linked to a termination signal and/or other regulatory element.

The term “homologs” as used herein refers to a gene related to a second gene by descent from a common ancestral DNA sequence. The term “homologs” may apply to the relationship between genes separated by the event of speciation (e.g., orthologs) or to the relationship between genes separated by the event of genetic duplication (e.g., paralogs).

As used herein, the term “orthologs” refers to genes from different species, but that have evolved from a common ancestral gene by speciation. Orthologs retain the same function in the course of evolution. Orthologs encode proteins having the same or similar functions. As used herein, the term “paralogs” refers to genes that are related by duplication within a genome. Paralogs usually have different functions or new functions, but these functions may be related.

The term “conserved region” or “conserved domain” as used herein refers to a region in heterologous polynucleotide or polypeptide sequences where there is a relatively high degree of sequence identity between the distinct sequences. The “conserved region” can be identified, for example, from the multiple sequence alignment using the Clustal W algorithm.

The term “cell” or “plant cell” as used herein refers to single cell, and also includes a population of cells. The population may be a pure population comprising one cell type. Likewise, the population may comprise more than one cell type. A plant cell within the meaning of the invention may be isolated (e.g., in suspension culture) or comprised in a plant tissue, plant organ or plant at any developmental stage.

The term “true breeding” as used herein refers to a variety of plant for a particular trait if it is genetically homozygous for that trait to the extent that, when the true-breeding variety is self-pollinated, a significant amount of independent segregation of the trait among the progeny is not observed.

The term “null segregant” as used herein refers to a progeny (or lines derived from the progeny) of a transgenic plant that does not contain the transgene due to Mendelian segregation.

The term “wild type” as used herein refers to a plant cell, seed, plant component, plant tissue, plant organ, or whole plant that has not been genetically modified or treated in an experimental sense.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

The term “syncytia site” as used herein refers to the feeding site formed in plant roots after nematode infestation. The site is used as a source of nutrients for the nematodes. A syncytium is the feeding site for cyst nematodes and giant cells are the feeding sites of root knot nematodes.

Crop plants and corresponding parasitic nematodes are listed in Index of Plant Diseases in the United States (U.S. Dept. of Agriculture Handbook No. 165, 1960); Distribution of Plant-Parasitic Nematode Species in North America (Society of Nematologists, 1985); and Fungi on Plants and Plant Products in the United States (American Phytopathological Society, 1989). For example, plant parasitic nematodes that are targeted by the present invention include, without limitation, cyst nematodes and root-knot nematodes. Specific plant parasitic nematodes which are targeted by the present invention include, without limitation, Heterodera glycines, Heterodera schachtii, Heterodera avenae, Heterodera oryzae, Heterodera cajani, Heterodera trifolii, Globodera pallida, G. rostochiensis, or Globodera tabacum, Meloidogyne incognita, M. arenaria, M. hapla, M. javanica, M. naasi, M. exigua, Ditylenchus dipsaci, Ditylenchus angustus, Radopholus similis, Radopholus citrophilus, Helicotylenchus multicinctus, Pratylenchus coffeae, Pratylenchus brachyurus, Pratylenchus vulnus, Paratylenchus curvitatus, Paratylenchus zeae, Rotylenchulus reniformis, Paratrichodorus anemones, Paratrichodorus minor, Paratrichodorus christiei, Anguina tritici, Bidera avenae, Subanguina radicicola, Hoplolaimus seinhorsti, Hoplolaimus Columbus, Hoplolaimus galeatus, Tylenchulus semipenetrans, Hemicycliophora arenaria, Rhadinaphelenchus cocophilus, Belonolaimus longicaudatus, Trichodorus primitivus, Nacobbus aberrans, Aphelenchoides besseyi, Hemicriconemoides kanayaensis, Tylenchorhynchus claytoni, Xiphinema americanum, Cacopaurus pestis, Heterodera zeae, Heterodera filipjevi and the like.

In one embodiment, the invention provides a nematode-resistant transgenic plant transformed with an expression vector comprising an isolated polynucleotide encoding at least one M. trunculata gene that encodes a CCP mature peptide that contains no more than four cysteine residues. Preferably, in this embodiment, the isolated polynucleotide has a sequence as defined in SEQ ID NO:1, 3, 5, 7, 9, 11, 13, 15 or 17. Alternatively, the polynucleotide encodes a polypeptide having a sequence as defined in SEQ ID NO:2, 4, 6, 8, 10, 12, 14, 16 or 18.

In accordance with the invention, the plant may be selected from the group consisting of monocotyledonous plants and dicotyledonous plants. The plant can be from a genus selected from the group consisting of maize, wheat, rice, barley, oat, rye, sorghum, banana, and ryegrass. The plant can be from a genus selected from the group consisting of pea, alfalfa, soybean, carrot, celery, tomato, potato, cotton, tobacco, pepper, oilseed rape, beet, cabbage, cauliflower, broccoli, lettuce and Arabidopsis thaliana.

The present invention also provides a plant, seed and parts from such a plant, and progeny plants from such a plant, including hybrids and inbreds. The invention also provides a method of plant breeding, e.g., to prepare a crossed fertile transgenic plant. The method comprises crossing a fertile transgenic plant comprising a particular expression vector of the invention with itself or with a second plant, e.g., one lacking the particular expression vector, to prepare the seed of a crossed fertile transgenic plant comprising the particular expression vector. The seed is then planted to obtain a crossed fertile transgenic plant. The plant may be a monocot. The crossed fertile transgenic plant may have the particular expression vector inherited through a female parent or through a male parent. The second plant may be an inbred plant. The crossed fertile transgenic may be a hybrid. Also included within the present invention are seeds of any of these crossed fertile transgenic plants.

The transgenic plants of the invention may be crossed with similar transgenic plants or with transgenic plants lacking the nucleic acids of the invention or with non-transgenic plants, using known methods of plant breeding, to prepare seeds. Further, the transgenic plant of the present invention may comprise, and/or be crossed to another transgenic plant that comprises one or more nucleic acids, thus creating a “stack” of transgenes in the plant and/or its progeny. The seed is then planted to obtain a crossed fertile transgenic plant comprising the nucleic acid of the invention. The crossed fertile transgenic plant may have the particular expression cassette inherited through a female parent or through a male parent. The second plant may be an inbred plant. The crossed fertile transgenic may be a hybrid. Also included within the present invention are seeds of any of these crossed fertile transgenic plants. The seeds of this invention can be harvested from fertile transgenic plants and be used to grow progeny generations of transformed plants of this invention including hybrid plant lines comprising the DNA construct.

“Gene stacking” can also be accomplished by transferring two or more genes into the cell nucleus by plant transformation. Multiple genes may be introduced into the cell nucleus during transformation either sequentially or in unison. In accordance with the invention, multiple M. trunculata genes encoding CCP mature peptides that comprise no more than four cysteine residues can be stacked to provide enhanced nematode resistance. These stacked combinations can be created by any method including but not limited to cross breeding plants by conventional methods or by genetic transformation. If the traits are stacked by genetic transformation, the M. trunculata genes can be combined sequentially or simultaneously in any order. For example if two genes are to be introduced, the two sequences can be contained in separate transformation cassettes or on the same transformation cassette. The expression of the sequences can be driven by the same or different promoters.

Another embodiment of the invention relates to an expression vector comprising a promoter operably linked to one or more polynucleotides of the invention, wherein expression of the polynucleotide confers increased nematode resistance to a transgenic plant. In one embodiment, the transcription regulatory element is a promoter capable of regulating constitutive expression of an operably linked polynucleotide. A “constitutive promoter” refers to a promoter that is able to express the open reading frame or the regulatory element that it controls in all or nearly all of the plant tissues during all or nearly all developmental stages of the plant. Constitutive promoters include, but are not limited to, the 35S CaMV promoter from plant viruses (Franck et al., Cell 21:285-294, 1980), the Nos promoter (An G. at al., The Plant Cell 3:225-233, 1990), the ubiquitin promoter (Christensen et al., Plant Mol. Biol. 12:619-632, 1992 and 18:581-8,1991), the MAS promoter (Velten et al., EMBO J. 3:2723-30, 1984), the maize H3 histone promoter (Lepetit et al., Mol Gen. Genet 231:276-85, 1992), the ALS promoter (WO96/30530), the 19S CaMV promoter (U.S. Pat. No. 5,352,605), the super-promoter (U.S. Pat. No. 5,955,646), the figwort mosaic virus promoter (U.S. Pat. No. 6,051,753), the rice actin promoter (U.S. Pat. No. 5,641,876), and the Rubisco small subunit promoter (U.S. Pat. No. 4,962,028).

In another embodiment, the transcription regulatory element is a regulated promoter. A “regulated promoter” refers to a promoter that directs gene expression not constitutively, but in a temporally and/or spatially manner, and includes both tissue-specific and inducible promoters. Different promoters may direct the expression of a gene or regulatory element in different tissues or cell types, or at different stages of development, or in response to different environmental conditions.

A “tissue-specific promoter” or “tissue-preferred promoter” refers to a regulated promoter that is not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of sequence. Suitable promoters include the napin-gene promoter from rapeseed (U.S. Pat. No. 5,608,152), the USP-promoter from Vicia faba (Baeumlein et al., Mol Gen Genet. 225(3):459-67, 1991), the oleosin-promoter from Arabidopsis (WO 98/45461), the phaseolin-promoter from Phaseolus vulgaris (U.S. Pat. No. 5,504,200), the Bce4-promoter from Brassica (WO 91/13980) or the legumin B4 promoter (LeB4; Baeumlein et al., Plant Journal, 2(2):233-9, 1992) as well as promoters conferring seed specific expression in monocot plants like maize, barley, wheat, rye, rice, etc. Suitable promoters to note are the 1pt2 or 1pt1-gene promoter from barley (WO 95/15389 and WO 95/23230) or those described in WO 99/16890 (promoters from the barley hordein-gene, rice glutelin gene, rice oryzin gene, rice prolamin gene, wheat gliadin gene, wheat glutelin gene, maize zein gene, oat glutelin gene, Sorghum kasirin-gene and rye secalin gene). Promoters suitable for preferential expression in plant root tissues include, for example, the promoter derived from corn nicotianamine synthase gene (US 20030131377) and rice RCC3 promoter (US 11/075,113). Suitable promoter for preferential expression in plant green tissues include the promoters from genes such as maize aldolase gene FDA (US 20040216189), aldolase and pyruvate orthophosphate dikinase (PPDK) (Taniguchi et. al., Plant Cell Physiol. 41(1):42-48, 2000).

Inducible promoters” refer to those regulated promoters that can be turned on in one or more cell types by an external stimulus, for example, a chemical, light, hormone, stress, or a nematode such as nematodes. Chemically inducible promoters are especially suitable if gene expression is wanted to occur in a time specific manner Examples of such promoters are a salicylic acid inducible promoter (WO 95/19443), a tetracycline inducible promoter (Gatz et al., Plant J. 2:397-404, 1992), the light-inducible promoter from the small subunit of Ribulose-1,5-bis-phosphate carboxylase (ssRUBISCO), and an ethanol inducible promoter (WO 93/21334). Also, suitable promoters responding to biotic or abiotic stress conditions are those such as the nematode inducible PRP1-gene promoter (Ward et al., Plant. Mol. Biol. 22:361-366, 1993), the heat inducible hsp80-promoter from tomato (U.S. Pat. No. 5,187,267), cold inducible alpha-amylase promoter from potato (WO 96/12814), the drought-inducible promoter of maize (Busk et. al., Plant J. 11:1285-1295, 1997), the cold, drought, and high salt inducible promoter from potato (Kirch, Plant Mol. Biol. 33:897-909, 1997) or the RD29A promoter from Arabidopsis (Yamaguchi-Shinozalei et. al., Mol. Gen. Genet. 236:331-340, 1993), many cold inducible promoters such as corl5a promoter from Arabidopsis (Genbank Accession No U01377), blt101 and blt4.8 from barley (Genbank Accession Nos AJ310994 and U63993), wcs120 from wheat (Genbank Accession No AF031235), mlipl5 from corn (Genbank Accession No D26563), bn115 from Brassica (Genbank Accession No U01377), and the wound-inducible pinII-promoter (European Patent No. 375091).

Of particular utility in the present invention are syncytia site preferred, or nematode feeding site induced, promoters, including, but not limited to promoters from the Mtn3-like promoter disclosed in PCT/EP2008/051328, the Mtn21-like promoter disclosed in PCT/EP2007/051378, the peroxidase-like promoter disclosed in PCT/EP2007/064356, the trehalose-6-phosphate phosphatase-like promoter disclosed in PCT/EP2007/063761 and the At5g12170-like promoter disclosed in PCT/EP2008/051329. All of the forgoing applications are incorporated herein by reference.

Yet another embodiment of the invention relates to a method of producing a nematode-resistant transgenic plant, wherein the method comprises the steps of: a) transforming a wild-type plant with an expression vector comprising a polynucleotide encoding a; and c) selecting transgenic plants for increased nematode resistance.

A variety of methods for introducing polynucleotides into the genome of plants and for the regeneration of plants from plant tissues or plant cells are known in, for example, Plant Molecular Biology and Biotechnology (CRC Press, Boca Raton, Fla.), chapter 6/7, pp. 71-119 (1993); White FF (1993) Vectors for Gene Transfer in Higher Plants; Transgenic Plants, vol. 1, Engineering and Utilization, Ed.: Kung and Wu R, Academic Press, 15-38; Jenes B et al. (1993) Techniques for Gene Transfer; Transgenic Plants, vol. 1, Engineering and Utilization, Ed.: Kung and R. Wu, Academic Press, pp. 128-143; Potrykus (1991) Annu Rev Plant Physiol Plant Molec Biol 42:205-225; Halford NG, Shewry PR (2000) Br Med Bull 56(1):62-73.

Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm ME et al., Bio/Technology. 8(9):833-9, 1990; Gordon-Kamm et al. Plant Cell 2:603, 1990), electroporation, incubation of dry embryos in DNA-comprising solution, and microinjection. In the case of these direct transformation methods, the plasmids used need not meet any particular requirements. Simple plasmids, such as those of the pUC series, pBR322, M13mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch RB et al. (1985) Science 225:1229. The Agrobacterium-mediated transformation is best suited to dicotyledonous plants but has also been adapted to monocotyledonous plants. The transformation of plants by Agrobacteria is described in, for example, White FF, Vectors for Gene Transfer in Higher Plants, Transgenic Plants, Vol. 1, Engineering and Utilization, edited by S. D. Kung and R. Wu, Academic Press, 1993, pp. 15 - 38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225.

The nucleotides described herein can be directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit high expression levels. In one embodiment, the nucleotides are inserted into a plastid targeting vector and transformed into the plastid genome of a desired plant host. Plants homoplasmic for plastid genomes containing the nucleotide sequences are obtained, and are preferentially capable of high expression of the nucleotides.

Plastid transformation technology is for example extensively described in U.S. Pat. Nos. 5,451,513, 5,545,817, 5,545,818, and 5,877,462 in WO 95/16783 and WO 97/32977, and in McBride et al. (1994) PNAS 91, 7301-7305.

The transgenic plants of the invention may be used in a method of controlling infestation of a crop by a plant nematode, which comprises the step of growing said crop from seeds comprising an expression vector comprising a promoter operably linked to a polynucleotide encoding at least one M. trunculata CCP mature peptide which comprises no more than four cysteine residues, wherein the expression vector is stably integrated into the genomes of the seeds.

The invention is further illustrated by the following examples, which are not to be construed in any way as imposing limitations upon the scope thereof.

Example 1: Cloning of MtCCP Genes from Medicago truncatula and Vector construction

Seeds of Medicago truncatula Jemalong A17 were germinated and cultivated in the green house. Genomic DNA was isolated from the shoots of these plants, and MtCCP genes were PCR amplified from this genomic DNA, using standard molecular biology techniques. The amplified product was ligated into a TOPO entry vector (Invitrogen, Carlsbad, Calif.).

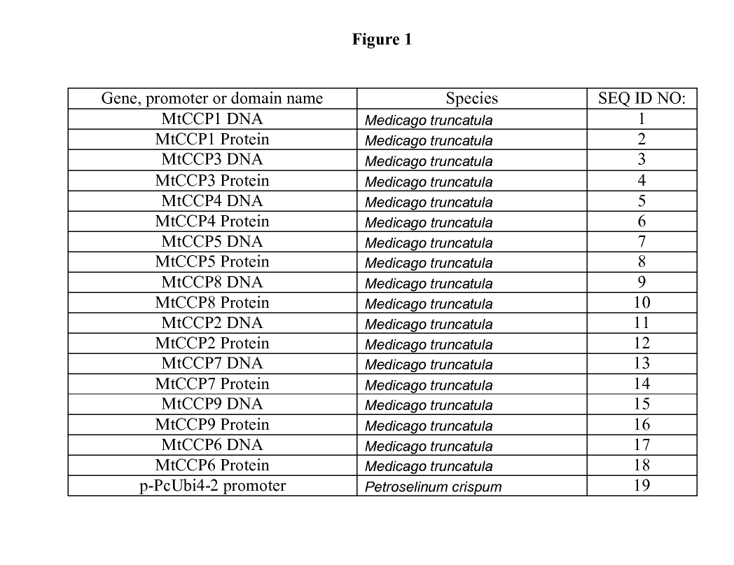

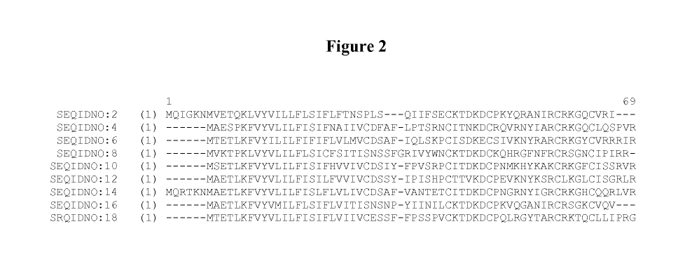

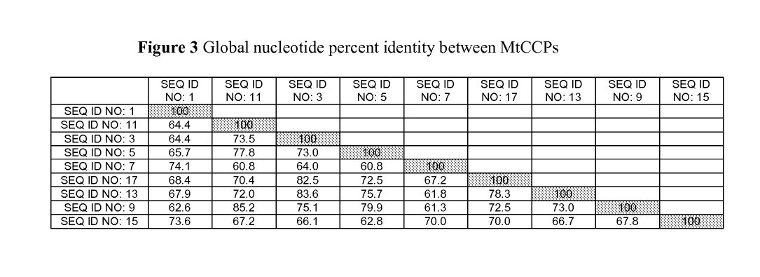

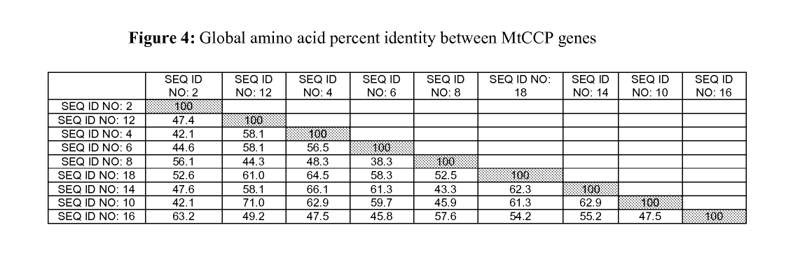

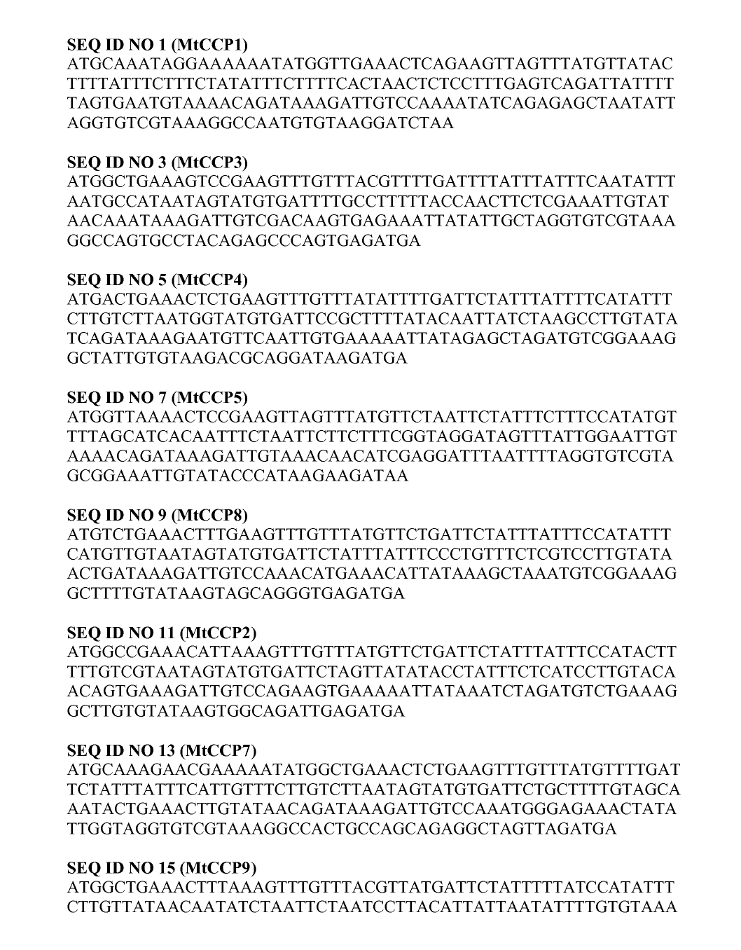

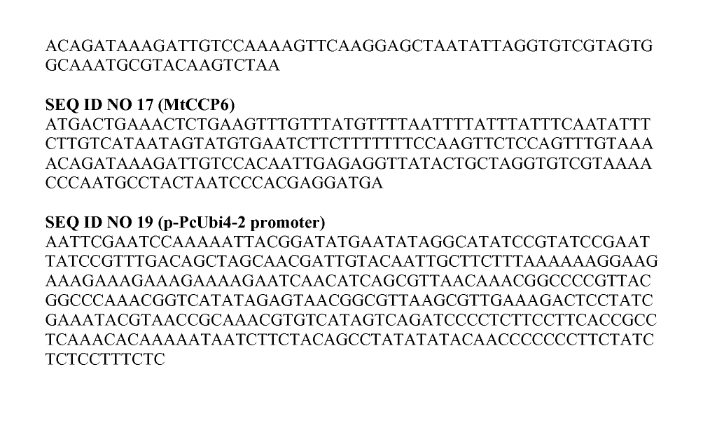

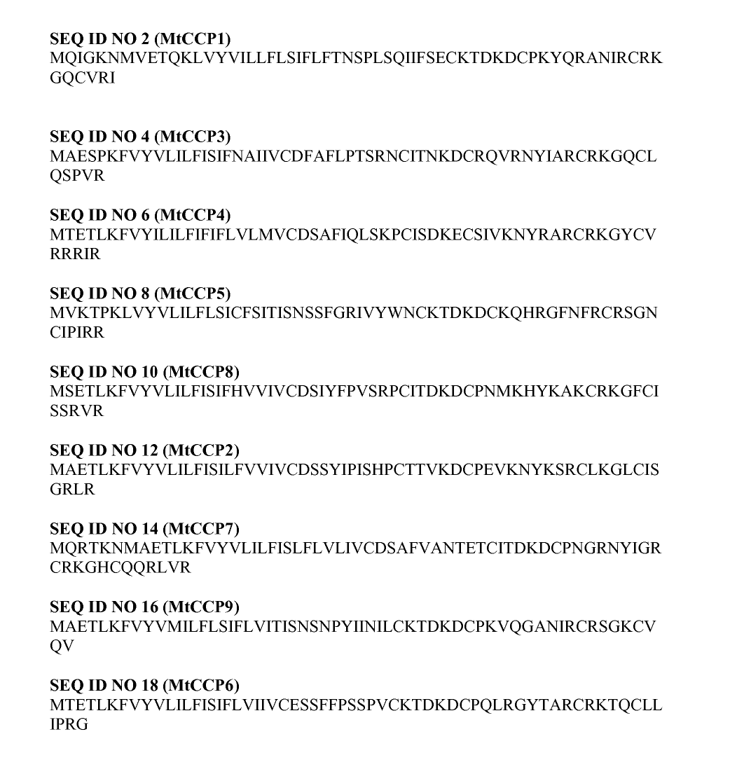

The cloned MtCCP genes were sequenced and subcloned into a plant expression vector containing a ubiquitin promoter from parsley (WO 03/102198; p-PcUbi4-2 promoter (SEQ ID NO:19) in FIG. 1). The selection marker for transformation was the mutated form of the acetohydroxy acid synthase (AHAS) selection gene (also referred to as AHAS2) from Arabidopsis thaliana (Sathasivan et al., Plant Phys. 97:1044-50, 1991), conferring resistance to the herbicide ARSENAL (Imazapyr, BASF Corporation, Mount Olive, N.J.). The expression of AHAS2 was driven by a ubiquitin promoter from parsley (WO 03/102198) (SEQ ID NO:19). Table 1 describes constructs containing the M. trunculata CCPs comprising no more than four cysteine residues in their mature peptides.

TABLE 1

| Vector Name | MtCCP gene | SEQ ID NO: of MtCCP genes |
|---|---|---|
| RTP1114-1 | MtCCP1 | 1 |
| RTP1116-3 | MtCCP3 | 3 |
| RTP1117-1 | MtCCP4 | 5 |
| RTP1118-1 | MtCCP5 | 7 |
| RTP1120-4 | MtCCP8 | 9 |
| RTP1115-4 | MtCCP2 | 11 |
| RTP1119-1 | MtCCP7 | 13 |
| RTP1121-2 | MtCCP9 | 15 |

### Example 2: Nematode Bioassay

A bioassay to assess nematode resistance conferred by the polynucleotides described herein was performed using a rooted plant assay system disclosed in commonly owned copending USSN 12/001,234. Transgenic roots are generated after transformation with the binary vectors described in Example 1. Multiple transgenic root lines are sub-cultured and inoculated with surface-decontaminated race 3 SCN second stage juveniles (J2) at the level of about 500 J2/well. Four weeks after nematode inoculation, the cyst number in each well is counted. For each transformation construct, the number of cysts per line is calculated to determine the average cyst count and standard error for the construct. The cyst count values for each transformation construct is compared to the cyst count values of an empty vector control tested in parallel to determine if the construct tested results in a reduction in cyst count. Two independent, biologically replicated experiments were run for each expression construct. Rooted explant cultures transformed with vectors RTP1114-1, RTP1116-3, RTP1117-1, RTP1118-1, and RTP1120-4 exhibited a general trend of reduced cyst numbers and female index relative to the known susceptible variety, Williams82.

1. A transgenic plant transformed with an expression vector comprising an isolated polynucleotide encoding at least one M. trunculata gene that encodes a CCP mature peptide that contains no more than four cysteine residues.

2. The transgenic plant of claim 1, wherein the isolated polynucleotide is selected from the group consisting of:
a) a polynucleotide having a sequence as defined in SEQ ID NO:1, 3, 5, 7, 9, 11, 13, 15 or 17; and
b) a polynucleotide encoding a polypeptide having a sequence as defined in SEQ ID NO:2, 4, 6, 8, 10, 12, 14, 16 or 18.

3. The plant of claim 1, wherein the plant is selected from the group consisting of maize, soybean, potato, cotton, oilseed rape, and wheat.

4. A seed which is true-breeding for at least one polynucleotide encoding at least one M. trunculata gene that encodes a CCP mature peptide that contains no more than four cysteine residues.

5. The seed of claim 1, wherein the isolated polynucleotide is selected from the group consisting of:
c) a polynucleotide having a sequence as defined in SEQ ID NO:1, 3, 5, 7, 9, 11, 13, 15 or 17; and
d) a polynucleotide encoding a polypeptide having a sequence as defined in SEQ ID NO:2, 4, 6, 8, 10, 12, 14, 16 or 18.

6. An expression vector comprising a promoter operably linked to an isolated polynucleotide encoding at least one M. trunculata gene that encodes a CCP mature peptide that contains no more than four cysteine residues.

7. The expression vector of claim 6, wherein the polynucleotide is selected from the group consisting of:
a) a polynucleotide having a sequence as defined in SEQ ID NO:1, 3, 5, 7, 9, 11, 13, 15 or 17; and
b) a polynucleotide encoding a polypeptide having a sequence as defined in SEQ ID NO:2, 4, 6, 8, 10, 12, 14, 16 or 18.

8. A method of producing a nematode-resistant transgenic plant, wherein the method comprises the steps of:
a) transforming a plant cell with an expression vector comprising an isolated polynucleotide encoding at least one M. trunculata gene that encodes a CCP mature peptide that contains no more than four cysteine residues;
b) generating transgenic plants from the transformed plant cell the transgenic plant; and
c) selecting transgenic plants with increased nematode resistance.

9. The method of claim 8, wherein the polynucleotide is selected from the group consisting of:
i) a polynucleotide having a sequence as defined in SEQ ID NO:1, 3, 5, 7, 9, 11, 13, 15 or 17; and
ii) a polynucleotide encoding a polypeptide having a sequence as defined in SEQ ID NO:2, 4, 6, 8, 10, 12, 14, 16 or 18.


SEQUENCE LISTING (19 sequences)

SEQ ID NO: 1 - DNA, 192 bp, Medicago truncatula
atgcaaataggaaaaaatatggttgaaactcagaagttagtttatgttatacttttatttctttctatatttcttttcactaactctcctttgagtcagattatttttagtgaatgtaaaacagataaagattgtccaaaatatcagagagctaatattaggtgtcgtaaaggccaatgtgtaaggatctaa

SEQ ID NO: 2 - protein, 63 aa, Medicago truncatula
MQIGKNMVETQKLVYVILLFLSIFLFTNSPLSQIIFSECKTDKDCPKYQRANIRCRKGQCVRI

SEQ ID NO: 3 - DNA, 189 bp, Medicago truncatula
atggctgaaagtccgaagtttgtttacgttttgattttatttatttcaatatttaatgccataatagtatgtgattttgcctttttaccaacttctcgaaattgtataacaaataaagattgtcgacaagtgagaaattatattgctaggtgtcgtaaaggccagtgcctacagagcccagtgagatga

SEQ ID NO: 4 - protein, 62 aa, Medicago truncatula
MAESPKFVYVLILFISIFNAIIVCDFAFLPTSRNCITNKDCRQVRNYIARCRKGQCLQSPVR

SEQ ID NO: 5 - DNA, 189 bp, Medicago truncatula
atgactgaaactctgaagtttgtttatattttgattctatttattttcatatttcttgtcttaatggtatgtgattccgcttttatacaattatctaagccttgtatatcagataaagaatgttcaattgtgaaaaattatagagctagatgtcggaaaggctattgtgtaagacgcaggataagatga

SEQ ID NO: 6 - protein, 62 aa, Medicago truncatula
MTETLKFVYILILFIFIFLVLMVCDSAFIQLSKPCISDKECSIVKNYRARCRKGYCVRRRIR

SEQ ID NO: 7 - DNA, 189 bp, Medicago truncatula
atggttaaaactccgaagttagtttatgttctaattctatttctttccatatgttttagcatcacaatttctaattcttctttcggtaggatagtttattggaattgtaaaacagataaagattgtaaacaacatcgaggatttaattttaggtgtcgtagcggaaattgtatacccataagaagataa

SEQ ID NO: 8 - protein, 62 aa, Medicago truncatula
MVKTPKLVYVLILFLSICFSITISNSSFGRIVYWNCKTDKDCKQHRGFNFRCRSGNCIPIRR

SEQ ID NO: 9 - DNA, 189 bp, Medicago truncatula
atgtctgaaactttgaagtttgtttatgttctgattctatttatttccatatttcatgttgtaatagtatgtgattctatttatttccctgtttctcgtccttgtataactgataaagattgtccaaacatgaaacattataaagctaaatgtcggaaaggcttttgtataagtagcagggtgagatga

SEQ ID NO: 10 - protein, 62 aa, Medicago truncatula
MSETLKFVYVLILFISIFHVVIVCDSIYFPVSRPCITDKDCPNMKHYKAKCRKGFCISSRVR

SEQ ID NO: 11 - DNA, 189 bp, Medicago truncatula
atggccgaaacattaaagtttgtttatgttctgattctatttatttccatactttttgtcgtaatagtatgtgattctagttatatacctatttctcatccttgtacaacagtgaaagattgtccagaagtgaaaaattataaatctagatgtctgaaaggcttgtgtataagtggcagattgagatga

SEQ ID NO: 12 - protein, 62 aa, Medicago truncatula
MAETLKFVYVLILFISILFVVIVCDSSYIPISHPCTTVKDCPEVKNYKSRCLKGLCISGRLR

SEQ ID NO: 13 - DNA, 207 bp, Medicago truncatula
atgcaaagaacgaaaaatatggctgaaactctgaagtttgtttatgttttgattctatttatttcattgtttcttgtcttaatagtatgtgattctgcttttgtagcaaatactgaaacttgtataacagataaagattgtccaaatgggagaaactatattggtaggtgtcgtaaaggccactgccagcagaggctagttagatga

SEQ ID NO: 14 - protein, 68 aa, Medicago truncatula
MQRTKNMAETLKFVYVLILFISLFLVLIVCDSAFVANTETCITDKDCPNGRNYIGRCRKGHCQQRLVR

SEQ ID NO: 15 - DNA, 180 bp, Medicago truncatula
atggctgaaactttaaagtttgtttacgttatgattctatttttatccatatttcttgttataacaatatctaattctaatccttacattattaatattttgtgtaaaacagataaagattgtccaaaagttcaaggagctaatattaggtgtcgtagtggcaaatgcgtacaagtctaa

SEQ ID NO: 16 - protein, 59 aa, Medicago truncatula
MAETLKFVYVMILFLSIFLVITISNSNPYIINILCKTDKDCPKVQGANIRCRSGKCVQV

SEQ ID NO: 17 - DNA, 189 bp, Medicago truncatula
atgactgaaactctgaagtttgtttatgttttaattttatttatttcaatatttcttgtcataatagtatgtgaatcttctttttttccaagttctccagtttgtaaaacagataaagattgtccacaattgagaggttatactgctaggtgtcgtaaaacccaatgcctactaatcccacgaggatga

SEQ ID NO: 18 - protein, 62 aa, Medicago truncatula
MTETLKFVYVLILFISIFLVIIVCESSFFPSSPVCKTDKDCPQLRGYTARCRKTQCLLIPRG

SEQ ID NO: 19 - DNA, 325 bp, Petroselinum crispum
aattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctc